(19) United States
(12) Patent Application Publication
(10) Pub. No.: US 2025/0384684 A1
(43) Pub. Date: Dec. 18, 2025

(54) METHOD FOR ESTIMATING WHEAT LEAF AREA INDEX (LAI) TO MITIGATE IMPACT OF LEAF CHLOROPHYLL CONTENT (LCC) AND RESIDUE-SOIL BACKGROUND

(71) Applicant: NANJING AGRICULTURAL UNIVERSITY, Nanjing (CN)

(72) Inventors: Xia YAO, Nanjing (CN); Yan Zhu, Nanjing (CN); Wei Li, Nanjing (CN); Dong Li, Nanjing (CN); Jinxin Tang, Nanjing (CN); Xinwen Xu, Nanjing (CN); Mingyu LV, Nanjing (CN); Caili Guo, Nanjing (CN); Hengbiao Zheng, Nanjing (CN); Tao Cheng, Nanjing (CN); Weixing Cao, Nanjing (CN); Chongya Jiang, Nanjing (CN)

(73) Assignee: NANJING AGRICULTURAL UNIVERSITY, Nanjing (CN)

(21) Appl. No.: 18/741,004

(22) Filed: Jun. 12, 2024

(30) Foreign Application Priority Data
Apr. 14, 2023 (CN) 202310400044.2

(51) Int. Cl. G06V 20/10; G01N 21/31; G01N 21/84; G01N 33/00; G06V 10/776; G06V 20/13

(52) U.S. Cl. CPC G06V 20/188; G01N 21/31; G01N 21/84; G01N 33/0098; G06V 10/776; G06V 20/13; G06V 20/194; G01N 2021/8466

(57) ABSTRACT

A method for estimating a wheat leaf area index (LAI) to mitigate the impact of the leaf chlorophyll content (LCC) and a residue-soil background, including the following steps: step one, acquiring data; step two, calculating a residue-soil adjusted red edge difference index, including: a, calculating an existing REDVI on the basis of the wheat canopy spectrum; b, calculating an existing REDVI on the basis of the field background spectrum; and c, combining RE1 and R bands of the wheat canopy multispectral curve to construct RSARE; step three, constructing a wheat LAI estimation model: and step four, checking the wheat LAI estimation model. The method can simultaneously mitigate the impact of the residue-soil background and LCC in the LAI estimation process. Besides, the wheat LAI estimation model constructed on the basis of the index can estimate the LAI at an early stage in a wheat production process.

### TECHNICAL FIELD

The present invention belongs to the field of satellite-scale rapid nondestructive monitoring of crop growth on the basis of a reflection spectrum, and particularly relates to a method for estimating wheat green leaf area index (LAI) to mitigate impact of leaf chlorophyll content (LCC) and a residue-soil background.

### BACKGROUND

A wheat leaf arca index (LAI) is a critical vegetation canopy structure variable and is also an important variable for evaluating crop population growth. Meanwhile, the acquisition of the LAI is helpful for predicting crop growth and the yield. Traditional methods of ground LAI measurement are labor and time consuming and limited to relatively small areas, while satellite remote-sensing image can acquire the LAI at a large scale. In fact, there are various methods for estimating the LAI using satellite remote-sensing information, such as a vegetation index (VI), machine learning, and a radiative transfer model. Among them, the VI obtained by spectral reflectance is widely used for an LAI retrieval due to convenience.

The universality and extrapolation of a VI-LAI model are important for the large-scale retrieval of the LAI. However, the VI-LAI relationship is generally altered by the impact of multiple confounding factors, including soil background, leaf chlorophyll content (LCC), and canopy structure. Thus, numerous VIs have been used to solve the above problem to increase the robustness of a VI-LAI estimation model. Among them, although the modified triangular vegetation index (MTVI2) can simultaneously alleviate the impact of the LCC and soil background, it is affected by the vegetation canopy structure like most non-red-edge VIs. On the other hand, although most red-edge VIs can effectively mitigate the impact of the canopy structure and soil background, and thus have higher accuracy in the LAI retrieval process, they are easily affected by the LCC.

Today, the estimation of wheat LAI faces a huge challenge during an early stage due to variable background caused by straw returning to field and field moisture variation. Generally, variation in the background changes two spectral properties: spectral shape and spectral brightness. Soil moisture variation primarily affects the spectral brightness, and therefore many existing VIs are used to mitigate the brightness variation caused by the moisture variation, including a differential vegetation index (DVI) and a soil-adjusted vegetation index (SAVI). In fact, the VIs minimize the noise from the spectral brightness variation by adding adjustment factors applied to red and near infrared bands. These adjustment factors are usually assumed to only compensate for the spectral brightness variation, and they are viewed as being nearly equivalent. Due to the limited assumptions, this approach cannot reduce the noise from changes in the spectral shape of the background.

To address the issue of the changes in the spectral shape of the background, previous studies have tried to obtain the relationship between the adjustment factors applied to the red and near infrared bands, and the corresponding regression line is usually called a soil line. However, errors from the estimation of the soil line will affect the accuracy of the VI-LAI retrieval model. Hence, many studies also have replaced a red band with a red edge band to mitigate the impact caused by the changes in the spectral shape. The corresponding VIs include a red edge difference vegetation index (REDVI), a modified red-edge soil-adjusted index (MRESAVI), and a soil-adjusted red-edge index (SARE). Unfortunately, the VI that incorporate the red edge band is affected by LCC variation. On the other hand, although a recent study proposes a spectral separation algorithm of soil and vegetation (3SV) to mitigate the impact caused by changes in the spectral shape of the background on the basis of the reflectance at 477 nm and 677 nm, the application of the method from a satellite platform remains challenging due to the atmospheric effects on the blue band. For the satellite platform, there is a need to further develop a broadband red edge VI for mitigating the impact of the background and LCC in the LAI retrieval process.

The issue of the background in crop monitoring is particularly challenging in the case of wheat. In rice-wheat rotation crop fields, rice residues are often returned to the wheat field, leading to variable field backgrounds comprising soil, residue and residue-soil mixtures. The rice residue causes changes in the spectral properties of the background, especially to the spectral shape, consequently affecting the soil line and the VI-LAI relationship. As reported by a recent study, the crop residue generally causes changes of wheat canopy spectra and obviously affects the VI-LAI model. In addition, the residue on the field surface also affects moisture conditions, and the stability of the VI-LAI model can be further influenced by the coupling effect of the moisture and the background.

With the development of remote-sensing technology, satellite remote-sensing platform-borne sensors can provide visible light-near infrared band, and can also provide red edge and shortwave infrared bands, thereby providing abundant spectral information for remote-sensing monitoring of land vegetation. Although Landsat 8-9 satellite can provide the visible light-near infrared band and shortwave infrared band, its application is limited in the observation of discrete crop fields in China due to its 30 m spatial resolution. Worldview-2 satellite and RapidEye satellite can provide the visible light-near infrared band and red edge band at the same time, but these commercial satellites is limited to a certain extent during application. In recent years, Sentinel-2 launched by the European Space Agency can provide multiple red edge bands and shortwave infrared bands on the basis of the visible light-near infrared bands. In fact, the Sentinel-2 red-edge band has been proved to improve the retrieval accuracy of the vegetation LAI.

### SUMMARY

The present invention aims to provide a wheat green LAI estimation model to mitigate the impact of the LCC and a residue-soil background so as to simultaneously mitigate the impact of the residue-soil background and LCC in the LAI estimation process. Besides, the wheat LAI estimation model constructed on the basis of the index can estimate the LAI at an early stage in a wheat production process.

In order to realize the above objective, the present invention provides the following technical solution: a method for estimating a wheat LAI to mitigate the impact of the LCC and a residue-soil background, comprising the following steps:
step one, data acquisition: based on a Sentinel-2 satellite image, acquiring a wheat canopy multispectral curve and a field background multispectral curve as a wheat-residue-soil spectrum and a residue-soil spectrum respectively, and synchronously measuring a wheat LAI to obtain modeling data and checking data;
step two, calculating a residue-soil adjusted red edge difference index, comprising:
a. calculating an existing REDVI on the basis of the wheat canopy spectrum:

$${REDVI}^{c} = {NIR}^{c} - RE {2}^{c}$$

wherein REDVI^{c} represents REDVI obtained by calculating the canopy spectrum, and NIR^{c} and RE2^{c} respectively represent NIR and RE2 band reflectance of the wheat canopy spectrum obtained by the Sentinel-2 image;
b. calculating an existing REDVI on the basis of the field background spectrum:

$${REDVI}^{b} = {NIR}^{b} - RE {2}^{b}$$

wherein REDVI^{b} represents REDVI obtained by calculating the background spectrum, and NIR^{b} and RE2^{b} respectively represent NIR and RE2 band reflectance of the wheat field spectrum obtained by the Sentinel-2 image; and
c. combining RE1 and R bands of the wheat canopy multispectral curve to construct RSARE:

$$RSARE = \frac{{REDVI}^{c} - {REDVI}^{b}}{0 \text{.2} - {REDVI}^{b}} \cdot \frac{RE {1}^{c}}{{R}^{c}} = \frac{{NIR}^{c} - RE {2}^{c} - {NIR}^{b} + RE {2}^{b}}{0.2 - {NIR}^{b} + RE {2}^{b}} \cdot \frac{RE {1}^{c}}{{R}^{c}} ;$$

step three, constructing a wheat LAI estimation model: on the basis of the modeling data, establishing a relationship between the RSARE and LAI using a binomial model fitting, determining binomial model coefficients a, b and c, and establishing the wheat LAI estimation model; and
step four, checking the wheat LAI estimation model: validating and testing the wheat LAI estimation model using measured data of independent years, validating performances of the obtained LAI estimation model under a dry residue-soil background and a wet residue-soil background respectively, and simultaneously also validating the stability of the obtained LAI estimation model locally applied in remote popularization and application.

Further, in step one, the data acquisition comes from different years and different ecological spots; and the acquired sample data is respectively used as a modeling data set, a validating data set and a testing data set. Specifically, the method for acquiring the wheat canopy multispectral curve and the field background multispectral curve is as follows: acquiring the Sentinel-2 satellite image before wheat emergence and the Sentinel-2 satellite image corresponding to each growth stage after the wheat emergence. The Sentinel-2 satellite image is preprocessed using Sen2Cor and Sen2Res issued by the European Space Agency: the Sentinel-2 satellite image is subjected to radiometric calibration and atmospheric correction using the Sen2Cor; and coarse resolution bands of the Sentinel-2 satellite image is downscaled using the Sen2Res so as to improve the spatial resolution of each band of the Sentinel-2 satellite image to 10 m. The multispectral curves in the preprocessed Sentinel-2 satellite image were extracted using GPS information of a wheat sampling point acquired by field investigation, namely the field background multispectral curve and the wheat canopy multispectral curve. A red edge area (665 nm-783 nm) of the wheat canopy multispectral curve and the field background multispectral curve extracted from the Sentinel-2 satellite image includes 4 band information: R, RE1, RE2 and NIR.

In addition to acquiring latitude and longitude information of the wheat sampling point using GPS in the field investigation, wheat is acquired by a statistical method and a method for measuring the wheat LAI is as follows: counting the number of wheat stem tillers in a square frame with the side length of 1 m*1 m, obtaining 30 wheat stem tillers, separating same according to organs, scanning the area of wheat leaves using a leaf area meter, and calculating the sum of the areas of all the wheat leaves in 1 m*1 m, namely the wheat LAI.

The leaf area meter is an LI-3000c leaf area meter manufactured by the LI-COR company in the United states.

Further, in step one, the data acquisition comprises:
a. acquiring GPS information: acquiring latitude and longitude information using a handheld GPS instrument in field investigation of a wheat sampling point;
b. acquiring LAI data: counting the number of wheat stem tillers in a square frame with the side length of 1 m*1 m, then obtaining 30 wheat stem tillers, separating same according to organs, scanning the area of wheat leaves using an LI-3000c leaf area meter, and calculating the sum of the areas of all the wheat leaves in 1 m*1 m, namely the wheat LAI;
c. acquiring the Sentinel-2 satellite image: acquiring Sentinel-2 image data of a corresponding area and a corresponding time, including the Sentinel-2 satellite image before wheat emergence used for acquiring the field background multispectral curve; and the Sentinel-2 satellite image corresponding to each growth period after the wheat emergence used for acquiring the field wheat canopy multispectral curve;
d. preprocessing the Sentinel-2 satellite image: firstly, subjecting the Sentinel-2 satellite image to radiometric calibration and atmospheric correction using Sen2Cor, then downscaling coarse resolution bands of the Sentinel-2 satellite image using Sen2Res so as to improve the spatial resolution of each band of the Sentinel-2 satellite image to 10 m; and
e. acquiring Sentinel-2 multispectral information: extracting multispectral information of corresponding pixels in the preprocessed Sentinel-2 satellite image using GPS information measured by field investigation of a ground sampling point to obtain the wheat canopy multispectral curve and the field background multispectral curve, wherein a red edge area of the wheat canopy multispectral curve and the field background multispectral curve extracted from the Sentinel-2 satellite image includes 4 band information: R, RE1, RE2 and NIR; and the wheat canopy multispectral curve and the field background multispectral curve together with the ground measured LAI form the modeling data and the checking data for constructing and validating the LAI estimation model.

Further, in step two, on the basis of a linear spectral mixture analysis, REDVI^{c} is divided into three components, including wheat (REDVI^{w}), residue-soil background (REDVI^{b}) and errors (e):

$${REDVI}^{c} = α {REDVI}^{w} + ( 1 - α ) R {EDVI}^{b} + e$$

wherein REDVI^{w} is obtained by calculating the canopy spectrum of pure wheat at the 100% coverage, and on the basis of the stimulated result of a radiative transfer model PROSAIL under maximum LAI and LCC, the result of REDVI^{w}=0.2 is obtained; α is the canopy closure factor and ranges from 0% to 100%; e represents the errors of the linear spectral mixture analysis in the decomposition of REDVI^{c}; and if e is assumed to be 0, α is:

$$α = \frac{{REDVI}^{c} - {REDVI}^{b}}{{REDVI}^{w} - {REDVI}^{b}} = \frac{{REDVI}^{c} - {REDVI}^{b}}{0 \text{.2} - {REDVI}^{b}}$$

further, by substituting e=0 and REDVI^{w}=0.2 further into the equation of the linear spectral mixture analysis, it can be obtained:

$${REDVI}^{c} - ( 1 - α ) {REDVI}^{b} = α \cdot {REDVI}^{w} = \frac{{REDVI}^{c} - {REDVI}^{b}}{0 \text{.2} - {REDVI}^{b}} \cdot 0.2$$

to further eliminate the impact of the obtained spectral variables on chlorophyll, reference is further made to the existing modified chlorophyll absorption ratio index:

$$MCARI = [ ( {r}_{700} - {r}_{670} ) - 0.2 ( {r}_{700} - {r}_{550} ) ] \cdot ( {r}_{700} / {r}_{670} ) = ( {DVI}_{700 , 670} - 0.2 \cdot {DVI}_{700 , 550} ) \cdot ( {r}_{700} / {r}_{670} )$$

wherein r represents the reflectance at a specific wavelength position and DVI is the difference between the reflectance at the two wavelengths specified by the subscripts; and since the first bracket of MCARI is similar to the REDVI^{c}−(1−α)REDVI^{b}, besides, the Sentinel-2 has corresponding bands separately located at 705 nm and 665 nm, which are close to the 700 nm and 670 nm in the second bracket of MCARI, and therefore, r₇₀₅/r₆₆₅ is further multiplied on the basis of REDVI^{c}−(1−α)REDVI^{b}:

$$[ {REDVI}^{c} - ( 1 - a ) {REDVI}^{b} ] \cdot ( RE {1}^{c} / {R}^{c} ) = a \cdot {REDVI}^{w} \cdot ( RE {1}^{c} / {R}^{c} ) = 0.2 \cdot \frac{{REDVI}^{c} - {REDVI}^{b}}{0 \text{.2} - {REDVI}^{b}} \cdot \frac{RE {1}^{c}}{{R}^{c}}$$

wherein RE1^{c} and R^{c} respectively represent the Sentinel-2 red edge band at 705 nm and the Sentinel-2 red band at 665 nm; and in the equation, the coefficient of 0.2 can be removed since it does not affect the response of the spectral variables to the LAI sensitivity so as to obtain a new index RSARE:

$$RSARE = \frac{{REDVI}^{c} - {REDVI}^{b}}{0.2 - {REDVI}^{b}} \cdot \frac{RE {1}^{c}}{{R}^{c}} = \frac{{NIR}^{c} - RE {2}^{c} - {NIR}^{b} + RE {2}^{b}}{0.2 - N 1 {R}^{b} + RE {2}^{b}} \cdot \frac{RE {1}^{c}}{{R}^{c}} .$$

Further, simulation data of a PROSAIL model is used for testing an RSARE-LAI relationship, and the RSARE is proved to be capable of mitigating the impact of the complex residue-soil background and LCC in an LAI retrieval process.

Further, in step three, the wheat LAI estimation model is established using a binomial model to fit the relationship between the RSARE and LAI:
LAI=−2.02×RSARE²+5.66×RSARE+0.31.

Further, in step four, the corresponding determination coefficient R², root mean square error (RMSE) and relative root mean square error (RRMSE) are calculated:

$${R}_{val}^{2} = {( \frac{{\sum }_{n = 1}^{N} ( {LAI}_{e , n} - \overline{{LAI}_{e}} ) ( {LAI}_{m , n} - \overline{{LAI}_{m}} )}{\sqrt{{\sum }_{n = 1}^{N} {( {LAI}_{e , n} - \overline{{LAI}_{e}} )}^{2}} \sqrt{{\sum }_{n = 1}^{N} {( {LAI}_{m , n} - \overline{{LAI}_{m}} )}^{2}}} )}^{2}$$

$${RMSE}_{val} = \sqrt{\frac{1}{N} {\sum }_{n = 1}^{N} {( {LAI}_{e , n} - {LAI}_{m , n} )}^{2}}$$

$${RRMSE}_{val} ( \% ) = \frac{100}{\overline{{LAI}_{m}}} \sqrt{\frac{1}{N} {\sum }_{n = 1}^{N} {( {LAI}_{e , n} - {LAI}_{m , n} )}^{2}}$$

wherein N denotes the number of samples in a data set, and LAI_{e,n}, LAI_{m,n}, L̅A̅I̅ₑ̅ and L̅A̅I̅ₘ̅ respectively represent the estimated LAI, the measured LAI, the mean value of the estimated LAI and the mean value of the measured LAI.

More specifically, the test results were Rᵥₐₗ²=0.76, RMSEᵥₐₗ=0.55 and RRMSEᵥₐₗ=20.71% in the dry residue-soil background; and the test results were Rᵥₐₗ²=0.56, RMSEᵥₐₗ=0.81 and RRMSEᵥₐₗ=24.92% in the wet residue-soil background.

Through the performance of the RSARE in estimating modeling and checking of the wheat LAI, the applicant finds that the RSARE and the LAI estimation model thereof can effectively mitigate the impact of the field complex residue-soil background, background moisture and LCC after straw returning to field in the wheat production process at an early growth stage of wheat, have higher fitting degree R² in the modeling process, and have higher R² and lower RMSE and RRMSE in the validation process. The spectral variable can effectively mitigate the impact of the mixed residue-soil background, background moisture and LCC.

Compared with the prior art, the present disclosure has the beneficial effects that:

By constructing the RSARE and the LAI estimation model thereof, the present invention can effectively mitigate the impact of the field complex residue-soil background, background moisture and LCC after straw returning to field in the wheat production process, especially at an early growth stage of wheat.

The present invention solves problems that the traditional soil vegetation index depends on the soil line and has limitation of eliminating the impact on a soil-dominated background, can effectively mitigate the impact of the wheat field complex residue-soil background and background moisture after straw returning to field, and can be used in real-time, nondestructive and accurate estimation of the regional-scale wheat LAI on the basis of the satellite platform.

### BRIEF DESCRIPTION OF THE DRAWINGS

FIG. 1 is a flow chart showing construction of RSARE, and a wheat LAI retrieval model at an early stage under the background of straw returning to field.

FIG. 2 shows a wheat canopy multispectral curve (i.e. wheat-residue-soil multispectral curve) and a field background multispectral curve (i.e. residue-soil multispectral curve).

FIG. 3 shows VI-LAI scatter plots drawn on the basis of simulated data with different residue-soil backgrounds (including soil, residue and residue-soil background) and LCCs (LCC=30 μg.cm⁻², 50 μg.cm⁻² and 70 μg.cm⁻²). The corresponding data are classified according to three LCC gradients to highlight the impact of different LCC levels on the VI-LAI relationship, including three levels LCC=30 μg.cm⁻², 50 μg.cm⁻² and 70 μg.cm⁻². The results show that the relationship between the non-red-edge VIs and LAI tends to be non-linear (FIG. 3a-e), and the residue-soil background has a moderate impact on the non-red-edge VI-LAI relationship. Among them, EVI has the best accuracy with R²=0.95 (FIG. 3c). In comparison, the traditional red-edge VIs are weakly correlated with the LAI (FIG. 3f-j), while the RSARE developed on the basis of REDVI has the highest correlation with the LAI (R²=0.96) (FIG. 3k). In conclusion, the RSARE can reduce the impact of the residue-soil background and LC an early LAI retrieval process. FIG. 3 also shows that the VI-LAI relationship changes according to the background and LCC. For example, in the DVI-LAI scatter plot (FIG. 3e), the scatter representing the residue background is obviously more curved than the scatter representing the soil background, demonstrating that the DVI-LAI relationship changes from linear to non-linear with the increasing of residue on the soil surface. Similarly, in the CI_{g}-LAI scatterplot (FIG. 3d), the scatter of the “*” symbol (i.e., LCC=70 μg/cm²) is more curved than the scatter of the “×” symbol (i.e., LCC=30 μg/cm²), indicating that the CI_{g}-LAI relationship changes from linear to non-linear with increasing the LCC.

FIG. 4 shows VI-LAI scatter plots drawn on the basis of simulated data with different soil backgrounds (including dry soil, semi-wet soil and wet soil backgrounds) and LCCs (LCC=30 μg.cm⁻², 50 μg.cm⁻² and 70 μg.cm⁻²). The results show that the non-red-edge VIs are less affected by the background with the corresponding R² between 0.71-0.93 (FIG. 4a-e). Among them, SAVI has the best performance with R²=0.93 (FIG. 4b). In comparison, the traditional red-edge VIs are more seriously affected by the LCC with the corresponding R² generally lower, between 0.53 and 0.82 (FIG. 4f-j). However, the RSARE obtained herein can simultaneously mitigate the impact of the background and LCC with the highest correlation with the LAI with R²=0.97 (FIG. 4k). In conclusion, the RSARE can be simultaneously suitable for the traditional soil background and the complex residue-soil background after straw returning to field, and has higher correlation with the LAI compared with the existing VIs.

FIG. 5 shows scatter plots of VI-LAI models calibrated with a dataset of dry soil and dry residue-soil backgrounds measured in 2021-2022. The traditional VI-LAI models can achieve similar accuracy under the dry soil background and the accuracy of the model calibration R_{cal}²=0.72-0.75 (FIG. 5a-j). However, when calibrated with data of different residue-soil backgrounds, the corresponding VI-LAI models have lower accuracy with R_{cal}²=0.59-0.69. Among them, REDVI has the highest accuracy (R_{cal}²=0.69), illustrating that it can reduce sensitivity to the residue-soil background. It is worth noting that although the traditional VI-LAI models can achieve better accuracy under a soil-dominated background (red dashed line in FIG. 5a-j) or a residue-soil background (blue dashed line in FIG. 5a-j), they have limited accuracy in both soil and residue-soil mixed backgrounds (black dashed line in FIG. 5a-j) with R_{cal}² between 0.61 and 0.68. In contrast, the RSARE-LAI model achieves the highest accuracy during the calibration with R_{cal}²=0.72 (FIG. 5k) and is the least affected by the background. In fact, the RSARE-LAI model performs best both in the soil background and the residue-soil background with the accuracy R_{cal}²=0.77 and R_{cal}²=0.69 respectively.

FIG. 6 shows scatter plots of VI-LAI models validated with a dataset of dry residue-soil background measured in 2021-2022. Due to the impact from the residue-soil background, the validation accuracy of the non-red-edge VI-LAI model is RMSE of 0.71-1.29 and RRMSE of 26.79%-48.73% (FIG. 6a-e). On the other hand, the traditional red-edge VIs produce higher accuracy than the non-red-edge VIs, with RMSE=0.59-0.77 and RRMSE=22.07%-29.03% respectively (FIG. 6f-j). The results illustrate that red-edge bands have the capability to reduce residue-soil noise. Overall, NDVI, CI_{g}, NDRE and CI_{RE} tend to overestimate the LAI due to the impact from the residue-soil background, while DVI, REDVI, EVI and MEVI generally underestimate the LAI, especially for high values of the LAI (LAI>2.0). Among all the VI-LAI models, the RSARE-LAI model performs most stably in different residue-soil backgrounds, with RMSE and RRMSE of 0.55 and 20.71% respectively.

FIG. 7 shows a performance comparison of traditional SARE-LAI and RSARE-LAI models with a data set of a wet residue-soil background measured in 2017-2018. The data points are coded by NDWI, which is used to indicate field moisture content.

### DETAILED DESCRIPTION

The present invention is described in detail below with reference to the accompanying drawings and specific examples.

### Example 1

The present example was performed on the basis of data of field investigation of different years and corresponding Senitnel-2 image data specifically shown in Table 1:

TABLE 1
Ground investigation and acquisition of Sentinel-2
satellite images at different growth stages

| Years& | Growth | Ground | Sentinel-2 image | Background | Sample |
|---|---|---|---|---|---|
| sites | stage | sampling date | acquisition date | conditions | number |
| 2017-2018 | Before | / | Nov. 10, 2017 | Dry residue-soil | 21 |
| Xinghua | emergence | | | | |
| | Tillering | Mar. 8-Mar. 10, 2018 | Mar. 10, 2018 | Wet residue-soil | |
| | stage | | | | |
| 2020-2021 | Before | / | Nov. 9, 2020 | Dry residue-soil | 33 |
| Xinghua | emergence | | | | |
| | Tillering | Mar. 12-Mar. 14, 2021 | Mar. 14, 2021 | Dry residue-soil | |
| | stage | | | | |
| 2021-2022 | Before | / | Dec. 4, 2021 | Dry soil and | 96 |
| Suzhou | emergence | | | residue-soil | |
| | Tillering | Feb. 24-Feb. 28, 2021 | Feb. 27, 2022 | Dry soil and | |
| | stage | | | residue-soil | |
| | | Mar. 8-Mar. 10, 2021 | Mar. 9, 2022 | Dry soil and | |
| | | | | residue-soil | |

The measured data of the field investigation in Suzhou in 2021-2022 was used as a modeling data set. The data set had the characteristics of better systematicness, more sample quantity and the like, and included samples of a pure soil background and a mixed residue-soil background, such that the effect of the obtained model in estimating LAI under different backgrounds can be tested.

The measured data of the field investigation in Xinghua in 2020-2021 was used as a validating data set. Compared with the modeling data, the validating data set had low requirement and less sample number, was influenced by environmental differences of different years and different ecological spots, and can be used for testing the universality of the obtained LAI estimation model in different geographic positions and different years.

The measured data of the investigation in Xinghua in 2017-2018 was used as a testing data set. Due to cloud and rain weather before satellite transit, the data set showed a wet mixed residue-soil background. Due to the shielding problem of the cloud layer, the data set only contained 21 effective samples. However, it can also be used for testing the performance of the obtained LAI estimation model under the residue-soil background with different moisture conditions.

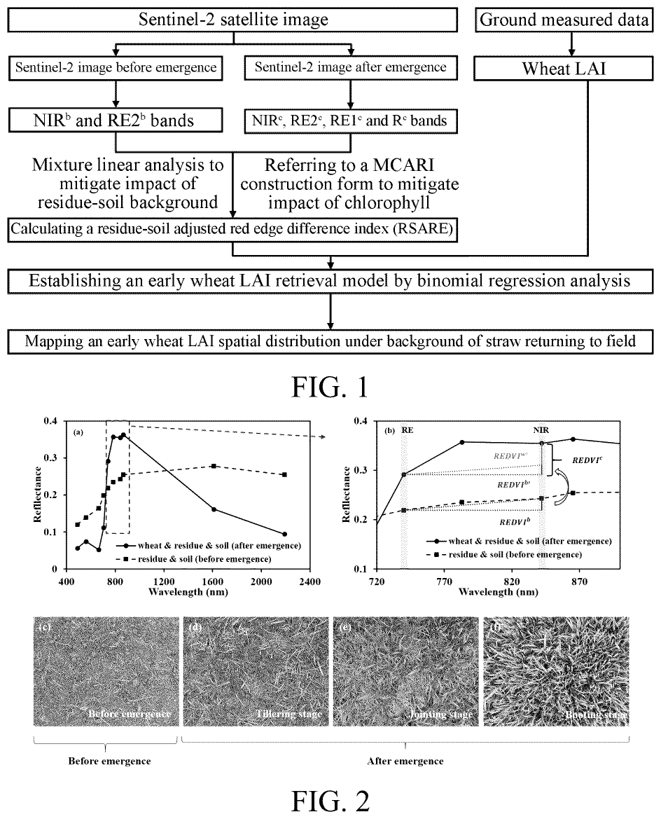

A method for estimating a wheat LAI to mitigate the impact of the LCC and residue-soil background specifically comprised the following steps:
step (1), acquiring data:
a. acquiring GPS information: latitude and longitude information was acquired using a handheld GPS instrument in field investigation of a wheat sampling point;
b. acquiring LAI data: the number of wheat stem tillers in a square frame with the side length of 1 m*1 m was counted, then 30 wheat stem tillers was obtained and separated according to organs, the area of wheat leaves was scanned using an LI-3000c leaf area meter (LI-COR company in the United states), and the sum of the areas of all the wheat leaves in 1 m*1 m was calculated, namely the wheat LAI;
c. acquiring the Sentinel-2 satellite image: Sentinel-2 image data of a corresponding arca and a corresponding time was acquired from an office network of the European Space Agency (https://scihub.copernicus.cu/dhus/#/home), including the Sentinel-2 satellite image before wheat emergence used for acquiring the field background multispectral curve; and the Sentinel-2 satellite image corresponding to each growth stage after the wheat emergence used for acquiring the field wheat canopy multispectral curve;
d. preprocessing the Sentinel-2 satellite image: firstly, the Sentinel-2 satellite image was subjected to radiometric calibration and atmospheric correction using Sen2Cor, then coarse resolution bands of the Sentinel-2 satellite image were downscaled using Sen2Res so as to improve the spatial resolution of each band of the Sentinel-2 satellite image to 10 m; and
e. acquiring Sentinel-2 multispectral information: multispectral information of corresponding pixels in the preprocessed Sentinel-2 satellite image was extracted using GPS information measured by field investigation of a ground sampling point to obtain the wheat canopy multispectral curve (i.e. wheat-residue-soil multispectral curve) and the field background multispectral curve (i.e. residue-soil multispectral curve) shown in FIG. 1, wherein a red edge area of the wheat canopy multispectral curve and the field background multispectral curve extracted from the Sentinel-2 satellite image included 4 band information: R, RE1, RE2 and NIR; and the wheat canopy multispectral curve and the field background multispectral curve together with the ground measured LAI formed the modeling data and the checking data for constructing and validating the LAI estimation model.
step (2), a novel residual-soil adjusted red edge difference index (RSARE) was constructed on the basis of the red edge area band of the field background spectrum before the wheat emergence and the red edge area band of the wheat field canopy spectrum at each growth stage after the wheat emergence:
a. a REDVI of the wheat canopy multispectral curve was calculated:

$${REDVI}^{c} = {NIR}^{c} - RE {2}^{c}$$

b. a REDVI of the field background multispectral curve was calculated:

$${REDVI}^{b} = {NIR}^{b} - RE {2}^{b}$$

c. RE1 and R bands of the wheat canopy multispectral curve were combined to construct RSARE:

$$RSARE = \frac{{REDVI}^{c} - {REDVI}^{b}}{0.2 - {REDVI}^{b}} \cdot \frac{RE {1}^{c}}{{R}^{c}} = \frac{{NIR}^{c} - RE {2}^{c} - {NIR}^{b} + RE {2}^{b}}{0.2 - {NIR}^{b} + RE {2}^{b}} \cdot \frac{RE {1}^{c}}{{R}^{c}} ;$$

step (3), on the basis of the modeling data, a wheat LAI estimation model was established using a binomial model to fit the relationship between the RSARE and LAI:
LAI=−2.02×RSARE²+5.66×RSARE+0.31; and
step (4), checking the wheat LAI estimation model: the wheat LAI estimation model was validated and tested using measured data of independent years, performances of the obtained LAI estimation model under a dry residue-soil background and a wet residue-soil background were validated respectively, and simultaneously the stability of the obtained LAI estimation model locally applied in remote popularization and application was also validated; and the corresponding determination coefficient R², root mean square error (RMSE) and relative root mean square error (RRMSE) were calculated:

$${R}_{v a l}^{2} = {( \frac{{\sum }_{n = 1}^{N} ( {LAI}_{e , n} − \overline{{LAI}_{e}} ) ( L A {I}_{m , n} − \overline{{LAI}_{m}} )}{\sqrt{{\sum }_{n = 1}^{N} {( LA {I}_{e , n} − \overline{{LAI}_{e}} )}^{2}} \sqrt{{\sum }_{n = 1}^{N} {( {LAI}_{m , n} − \overline{{LAI}_{m}} )}^{2}}} )}^{2}$$

$${RMSE}_{v a l} = \sqrt{\frac{1}{N} {\sum }_{n = 1}^{N} {( {LAI}_{e , n} - {LAI}_{m , n} )}^{2}}$$

$$RRMS {E}_{v a l} ( \% ) = \frac{100}{\overline{{LAI}_{m}}} \sqrt{\frac{1}{N} {\sum }_{n = 1}^{N} {( {LAI}_{e , n} - {LAI}_{m , n} )}^{2}}$$

wherein N denoted the number of samples in a data set, and LAI_{e,n}, LAI_{m,n}, L̅A̅I̅ₑ̅ and L̅A̅I̅ₘ̅ respectively represented the estimated LAI, the measured LAI, the mean value of the estimated LAI and the mean value of the measured LAI.

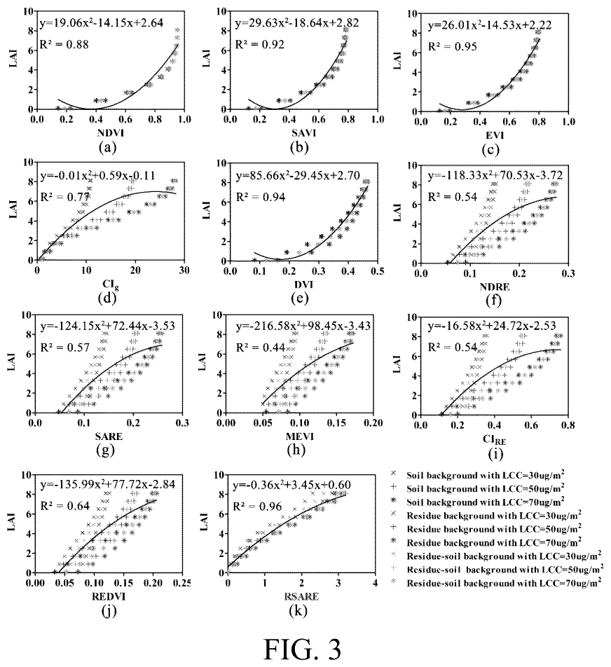

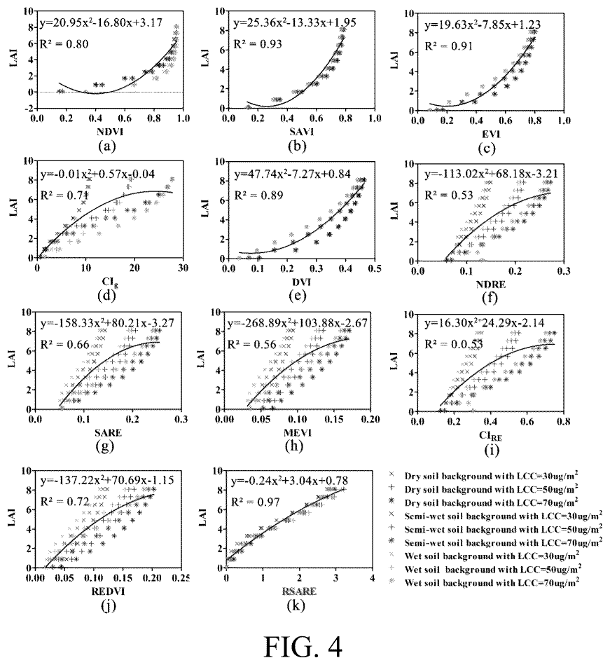

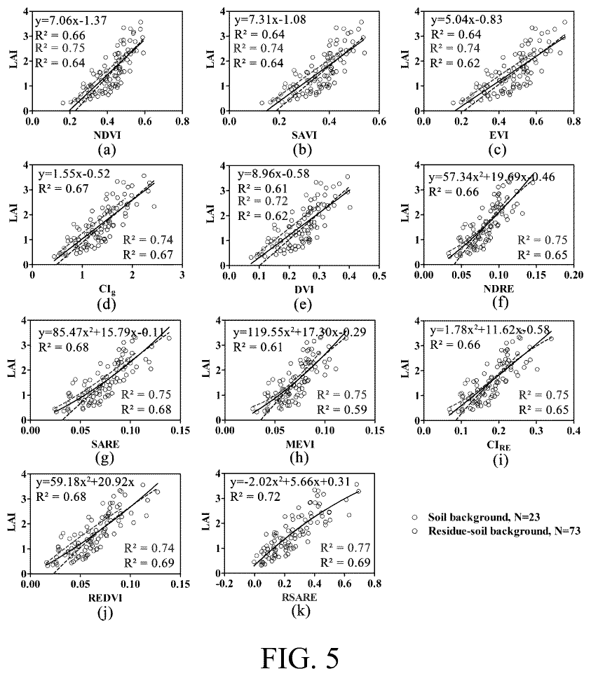

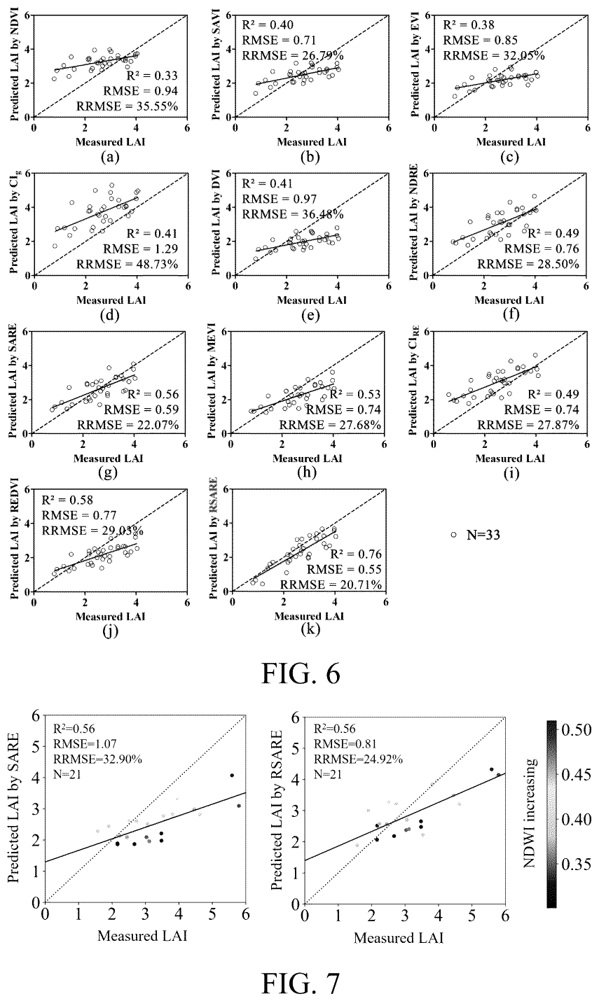

The test results were shown in FIGS. 2-6. The simulated data validation showed that the RSARE can mitigate the impact of the traditional soil background, the mixed residue-soil background after straw returning to field and the LCC, and had the best effect compared to the existing VIs. In the process of model construction, the RSARE obtained the best modeling accuracy specifically with R²=0.72 due to the mitigation of the mixed residue-soil background. In the validation result under the dry residue-soil background, the RSARE obtained the best performance compared to the existing VIs with the specific accuracy of Rᵥₐₗ²=0.76, RMSEᵥₐₗ=0.55 and RRMSEᵥₐₗ=20.71%. In the validation result under the wet residue-soil background, the RSARE was also superior to due to the traditional VIs with the specific accuracy of Rᵥₐₗ²=0.56, RMSEᵥₐₗ=0.81 and RRMSEᵥₐₗ=24.92%.

The RSARE constructed by the present example can eliminate the impact of the wheat field complex background after straw returning to field, including the traditional soil background, the mixed residue-soil background after straw returning to field and the corresponding background moisture, and simultaneously can also mitigate the impact of the LCC, thereby comprehensively improving the wheat LAI estimation, particularly improving the wheat LAI estimation at an early growth stage. The obtained LAI estimation model was robust.

The above shows and describes the basic principles, main features, and advantages of the present invention. Those skilled in the art should understand that the above examples are not intended to limit the present invention in any form, and that any technical solutions obtained by means of equivalent replacement should fall within the protection scope of the present invention. Other parts not mentioned in the present invention are the same as those in the prior art or can be implemented by the prior art.

1. A method for estimating a wheat leaf area index (LAI) to mitigate the impact of the leaf chlorophyll content (LCC) and a residue-soil background, comprising the following steps:
step one, acquiring data: based on a Sentinel-2 satellite image, acquiring a wheat canopy multispectral curve and a field background multispectral curve as a wheat-residue-soil spectrum and a residue-soil spectrum respectively, and synchronously measuring a wheat LAI to obtain modeling data and checking data;
step two, calculating a residue-soil adjusted red edge difference index, comprising:
a. calculating an existing REDVI on the basis of the wheat canopy spectrum:
$${REDVI}^{c} = {NIR}^{c} - RE {2}^{c}$$
wherein REDVI^{c} represents REDVI obtained by calculating the canopy spectrum, and NIR^{c} and RE2^{c} respectively represent NIR and RE2 band reflectance of the wheat canopy spectrum obtained by the Sentinel-2 image;
b. calculating an existing REDVI on the basis of the field background spectrum:
$${REDVI}^{b} = {NIR}^{b} - RE {2}^{b}$$
wherein REDVI^{b} represents REDVI obtained by calculating the background spectrum, and NIR^{b} and RE2^{b} respectively represent NIR and RE2 band reflectance of the wheat field spectrum obtained by the Sentinel-2 image; and
c. combining RE1 and R bands of the wheat canopy multispectral curve to construct RSARE:
$$RSARE = \frac{{REDVI}^{c} - {REDVI}^{b}}{0.2 - {REDVI}^{b}} \cdot \frac{RE {1}^{c}}{{R}^{c}} = \frac{{NIR}^{c} - RE {2}^{c} - {NIR}^{b} + RE {2}^{b}}{0.2 - {NIR}^{b} + RE {2}^{b}} \cdot \frac{RE {1}^{c}}{{R}^{c}} ;$$
step three, constructing a wheat LAI estimation model: on the basis of the modeling data, establishing a relationship between the RSARE and LAI using a binomial model fitting, determining binomial model coefficients a, b and c, and establishing the wheat LAI estimation model; and specifically, establishing the wheat LAI estimation model using the binomial model to fit the relationship between the RSARE and LAI:
$$LAI = - 2.02 \times R S A R {E}^{2} + 5.66 \times R S A R E + 0.31 ;$$
step four, checking the wheat LAI estimation model: validating and testing the wheat LAI estimation model using measured data of independent years, validating performances of the obtained LAI estimation model under a dry residue-soil background and a wet residue-soil background respectively, and simultaneously also validating the stability of the obtained LAI estimation model locally applied in remote popularization and application.

2. The method for estimating a wheat LAI to mitigate the impact of the LCC and a residue-soil background according to claim 1, wherein in step one, the data acquisition comes from different years and different ecological spots; and the acquired sample data is respectively used as a modeling data set, a validating data set and a testing data set.

3. The method for estimating a wheat LAI to mitigate the impact of the LCC and a residue-soil background according to claim 1, wherein in step one, the data acquisition comprises:
a. acquiring GPS information: acquiring latitude and longitude information using a handheld GPS instrument in field investigation of a wheat sampling point;
b. acquiring LAI data: counting the number of wheat stem tillers in a square frame with the side length of 1 m*1 m, then obtaining 30 wheat stem tillers, separating same according to organs, scanning the area of wheat leaves using an LI-3000c leaf area meter, and calculating the sum of the areas of all the wheat leaves in 1 m*1 m, namely the wheat LAI;
c. acquiring the Sentinel-2 satellite image: acquiring Sentinel-2 image data of a corresponding area and a corresponding time, comprising the Sentinel-2 satellite image before wheat emergence used for acquiring the field background multispectral curve; and the Sentinel-2 satellite image corresponding to each growth stage after the wheat emergence used for acquiring the field wheat canopy multispectral curve;
d. preprocessing the Sentinel-2 satellite image: firstly, subjecting the Sentinel-2 satellite image to radiometric calibration and atmospheric correction using Sen2Cor, then downscaling coarse resolution bands of the Sentinel-2 satellite image using Sen2Res so as to improve the spatial resolution of each band of the Sentinel-2 satellite image to 10 m; and
e. acquiring Sentinel-2 multispectral information: extracting multispectral information of corresponding pixels in the preprocessed Sentinel-2 satellite image using GPS information measured by field investigation of a ground sampling point to obtain the wheat canopy multispectral curve and the field background multispectral curve, wherein a red edge area of the wheat canopy multispectral curve and the field background multispectral curve extracted from the Sentinel-2 satellite image comprises 4 band information: R, RE1, RE2 and NIR; and the wheat canopy multispectral curve and the field background multispectral curve together with the ground measured LAI form the modeling data and the checking data for constructing and validating the LAI estimation model.

4. The method for estimating a wheat LAI to mitigate the impact of the LCC and a residue-soil background according to claim 1, wherein in step two, on the basis of a linear spectral mixture analysis, REDVI^{c} is divided into three components, comprising wheat (REDVI^{w}), residue-soil background (REDVI^{b}) and errors (e):
$${REDVI}^{c} = α {REDVI}^{w} + ( 1 - α ) {REDVI}^{b} + e$$
wherein REDVI^{w} is obtained by calculating the canopy spectrum of pure wheat at the 100% coverage, and on the basis of the stimulated result of a radiative transfer model PROSAIL under maximum LAI and LCC, the result of REDVI^{w}=0.2 is obtained; α is the canopy closure factor and ranges from 0% to 100%; e represents the errors of the linear spectral mixture analysis in the decomposition of REDVI^{c}; and if e is assumed to be 0, α is:
$$α = \frac{{REDVI}^{c} - {REDVI}^{b}}{{REDVI}^{w} - {REDVI}^{b}} = \frac{{REDVI}^{c} - {REDVI}^{b}}{02 - {REDVI}^{b}}$$
further, by substituting e=0 and REDVI^{w}=0.2 further into the equation of the linear spectral mixture analysis, it can be obtained:
$${REDVI}^{c} - ( 1 - α ) {REDVI}^{b} = α \cdot {REDVI}^{w} = \frac{{REDVI}^{c} - {REDVI}^{b}}{02 - {REDVI}^{b}} \cdot 0.2$$
to further eliminate the impact of the obtained spectral variables on chlorophyll, reference is further made to the existing modified chlorophyll absorption ratio index:
$$\begin{matrix}MCARI = [ ( {r}_{700} - {r}_{670} ) - 0.2 ( {r}_{700} - {r}_{550} ) ] \cdot ( {r}_{700} / {r}_{670} ) \\ = ( {DVI}_{700 , 670} - 0.2 \cdot {DVI}_{700 , 550} ) \cdot ( {r}_{700} / {r}_{670} )\end{matrix}$$
wherein r represents the reflectance at a specific wavelength position and DVI is the difference between the reflectance at the two wavelength positions specified by the subscripts; and since the first bracket of MCARI is similar to the REDVI^{c}−(1−α)REDVI^{b}, besides, the Sentinel-2 has corresponding bands separately located at 705 nm and 665 nm, which are close to the 700 nm and 670 nm in the second bracket of MCARI, and therefore, r₇₀₅/r₆₆₅ is further multiplied on the basis of REDVI^{c}−(1−α)REDVI^{b}:
$$[ {REDVI}^{c} - ( 1 - a ) {REDVI}^{b} ] \cdot ( RE {1}^{c} / {R}^{c} ) = a \cdot {REDVI}^{w} \cdot ( RE {1}^{c} / {R}^{c} ) = 0.2 \cdot \frac{{REDVI}^{c} - {REDVI}^{b}}{02 - {REDVI}^{b}} \cdot \frac{RE {1}^{c}}{{R}^{c}}$$
wherein RE1^{c} and R^{c} respectively represent the Sentinel-2 red edge band at 705 nm and the Sentinel-2 red band at 665 nm; and in the equation, the coefficient of 0.2 can be removed since it does not affect the response of the spectral variables to the LAI sensitivity so as to obtain a new index RSARE:
$$RSARE = \frac{{REDVI}^{c} - {REDVI}^{b}}{0.2 - {REDVI}^{b}} \cdot \frac{RE {1}^{c}}{{R}^{c}} = \frac{{NIR}^{c} - RE {2}^{c} - {NIR}^{b} + RE {2}^{b}}{0.2 - {NIR}^{b} + RE {2}^{b}} \cdot \frac{{REI}^{c}}{{R}^{c}} .$$

5. The method for estimating a wheat LAI to mitigate the impact of the LCC and a residue-soil background according to claim 1, wherein simulation data of a PROSAIL model is used for testing an RSARE-LAI relationship, and the RSARE is proved to be capable of mitigating the impact of the complex residue-soil background and the LCC in an LAI retrieval process.

6. The method for estimating a wheat LAI to mitigate the impact of the LCC and a residue-soil background according to claim 1, wherein in step four, the corresponding determination coefficient R², root mean square error (RMSE) and relative root mean square error (RRMSE) are calculated:
$${R}_{v a l}^{2} = {( \frac{{\sum }_{n = 1}^{N} ( {LAI}_{e , n} − \overline{{LAI}_{e}} ) ( L A {I}_{m , n} − \overline{{LAI}_{m}} )}{\sqrt{{\sum }_{n = 1}^{N} {( LA {I}_{e , n} − \overline{{LAI}_{e}} )}^{2}} \sqrt{{\sum }_{n = 1}^{N} {( {LAI}_{m , n} − \overline{{LAI}_{m}} )}^{2}}} )}^{2}$$
$${RMSE}_{v a l} = \sqrt{\frac{1}{N} {\sum }_{n = 1}^{N} {( {LAI}_{e , n} - {LAI}_{m , n} )}^{2}}$$
$$RRMS {E}_{v a l} ( \% ) = \frac{100}{\overline{{LAI}_{m}}} \sqrt{\frac{1}{N} {\sum }_{n = 1}^{N} {( {LAI}_{e , n} - {LAI}_{m , n} )}^{2}}$$
wherein N denotes the number of samples in a data set, and LAI_{e,n}, LAI_{m,n}, L̅A̅I̅ₑ̅ and L̅A̅I̅ₘ̅ respectively represent the estimated LAI, the measured LAI, the mean value of the estimated LAI and the mean value of the measured LAI.

7. The method for estimating a wheat LAI to mitigate the impact of the LCC and a residue-soil background according to claim 2, wherein in step one, the data acquisition comprises:
a. acquiring GPS information: acquiring latitude and longitude information using a handheld GPS instrument in field investigation of a wheat sampling point;
b. acquiring LAI data: counting the number of wheat stem tillers in a square frame with the side length of 1 m*1 m, then obtaining 30 wheat stem tillers, separating same according to organs, scanning the area of wheat leaves using an LI-3000c leaf area meter, and calculating the sum of the areas of all the wheat leaves in 1 m*1 m, namely the wheat LAI;
c. acquiring the Sentinel-2 satellite image: acquiring Sentinel-2 image data of a corresponding area and a corresponding time, comprising the Sentinel-2 satellite image before wheat emergence used for acquiring the field background multispectral curve; and the Sentinel-2 satellite image corresponding to each growth stage after the wheat emergence used for acquiring the field wheat canopy multispectral curve;
d. preprocessing the Sentinel-2 satellite image: firstly, subjecting the Sentinel-2 satellite image to radiometric calibration and atmospheric correction using Sen2Cor, then downscaling coarse resolution bands of the Sentinel-2 satellite image using Sen2Res so as to improve the spatial resolution of each band of the Sentinel-2 satellite image to 10 m; and
e. acquiring Sentinel-2 multispectral information: extracting multispectral information of corresponding pixels in the preprocessed Sentinel-2 satellite image using GPS information measured by field investigation of a ground sampling point to obtain the wheat canopy multispectral curve and the field background multispectral curve, wherein a red edge area of the wheat canopy multispectral curve and the field background multispectral curve extracted from the Sentinel-2 satellite image comprises 4 band information: R, RE1, RE2 and NIR; and the wheat canopy multispectral curve and the field background multispectral curve together with the ground measured LAI form the modeling data and the checking data for constructing and validating the LAI estimation model.

8. The method for estimating a wheat LAI to mitigate the impact of the LCC and a residue-soil background according to claim 4, wherein simulation data of a PROSAIL model is used for testing an RSARE-LAI relationship, and the RSARE is proved to be capable of mitigating the impact of the complex residue-soil background and the LCC in an LAI retrieval process.
